(19) United States
(12) Patent Application Publication
(10) Pub. No.: US 2017/0177795 A1
(43) Pub. Date: Jun. 22, 2017

(54) METHOD AND SYSTEM FOR VISUALIZATION OF PATIENT HISTORY

(71) Applicant: KONINKLIJKE PHILIPS N.V., EINDHOVEN (NL)

(72) Inventors: THUSITHA DANANJAYA DE SILVA MABOTUWANA, BOTHELL, WA (US); YUECHEN QIAN, LEXINGTON, MA (US); JOHANNES BUURMAN, 's-Hertogenbosch (NL)

(21) Appl. No.: 15/302,024

(22) PCT Filed: Apr. 8, 2015
(86) PCT No.: PCT/IB2015/052526

§ 371 Date: Oct. 5, 2016

Related U.S. Application Data
(60) Provisional application No. 61/980,768, filed on Apr. 17, 2014.

(51) Int. Cl. G06F 19/00; A61B 5/01; A61B 5/055; G06F 17/27; A61B 6/00; A61B 8/08; G06T 7/00; A61B 5/00; A61B 6/03

(52) U.S. Cl. CPC G06F 19/321; A61B 5/0095; A61B 5/015; A61B 5/055; A61B 6/032; A61B 6/037; A61B 6/5217; A61B 8/0883; A61B 8/5223; G06T 7/0012; G06F 17/2705; G06T 2207/10081; G06T 2207/10116; G06T 2207/10088; G06T 2207/10132; G06T 2207/10104

(57) ABSTRACT

A system and method for receiving a plurality of reports, each of the reports describing a corresponding one of a plurality of medical imaging studies of a patient, extracting, from each of the reports, a corresponding characteristic, identifying a subset of the reports based on a similarity of the characteristics of the reports comprising the subset and generating a visualization of a portion of a patient history for the patient, the portion comprising the subset of the reports.

### BACKGROUND

Prior to conducting a radiology study, a radiologist may examine one or more relevant prior imaging studies in order to establish proper context for the current study. Establishing context may be a non-trivial task, particularly in the case of cancer patients, whose histories may include related findings across multiple clinical episodes. Existing radiology equipment provides a patient's past studies along a basic timeline, which may enhance the difficulty of establishing proper context.

### BRIEF DESCRIPTION OF THE DRAWINGS

FIG. 1 illustrates two prior art visualizations of a history of patient imaging studies.

FIG. 2 schematically illustrates a system for visualization of patient history according to an exemplary embodiment.

FIG. 3 shows an exemplary method for visualization of patient history using a system such as the exemplary system of FIG. 2.

FIG. 4 shows a first exemplary visualization of patient history that may be generated by the exemplary system of FIG. 2 and the exemplary method of FIG. 3.

FIG. 5 shows a second exemplary visualization of patient history that may be generated by the exemplary system of FIG. 2 and the exemplary method of FIG. 3.

FIG. 6 shows a third exemplary visualization of patient history that may be generated by the exemplary system of FIG. 2 and the exemplary method of FIG. 3.

FIG. 7 shows a fourth exemplary visualization of patient history that may be generated by the exemplary system of FIG. 2 and the exemplary method of FIG. 3.

### DETAILED DESCRIPTION

The exemplary embodiments may be further understood with reference to the following description and the related appended drawings, wherein like elements are provided with the same reference numerals. Specifically, the exemplary embodiments relate to methods and systems for visualization of complex patient histories of imaging studies.

Radiologists typically must familiarize themselves with a large number of prior studies in order to diagnose and treat patients in an effective manner. The use of prior studies is required in order to establish proper context for a current study. In particular, cancer patients may frequently undergo imaging studies, resulting in a large number of prior studies to be reviewed by a radiologist. The designation “radiologist” is used throughout this description to refer to the individual who is reviewing a patient's medical records, but it will be apparent to those of skill in the art that the individual may alternatively be any other appropriate user, such as a doctor, nurse, or other medical professional.

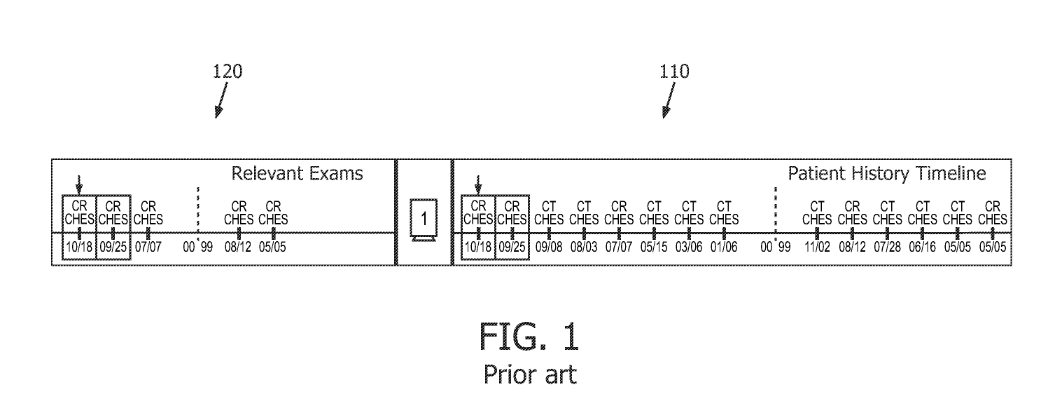

Prior art solutions typically display previous studies along a basic timeline. FIG. 1 shows two such prior art timelines of studies. In some solutions, all studies are shown along a single timeline. Timeline 110, on the right hand side of FIG. 1, presents such a display of studies. In the timeline 110, all prior studies for a given patient are shown. The timeline 110 includes CT studies and CR studies of a patient's chest over a time period, but those of skill in the art will understand that this is only exemplary, and that other timelines may include a broader variety of types of studies of different regions of the patient's body.

At most, prior solutions may group all studies of the same type (e.g., all studies having the same modality and body part) along a more focused timeline. Timeline 120, on the left hand side of FIG. 1, includes a subset of the studies shown in timeline 110. Specifically, timeline 120 includes CR studies of the patient's chest over the same period as timeline 110, while omitting the CT studies shown in timeline 110. It will be apparent that the selection of CR chest studies is only exemplary and that different subsets may be possible.

The process of reviewing prior studies typically involves opening one or more prior reports, which typically include images and accompanying text in a narrative form. However, the generalized views presented by the prior art as shown in FIG. 1 provide minimal assistance to the radiologist in selecting the prior reports to review. Further, the prior art timelines themselves provide no particular guidance to the radiologist in establishing proper context for a current study.

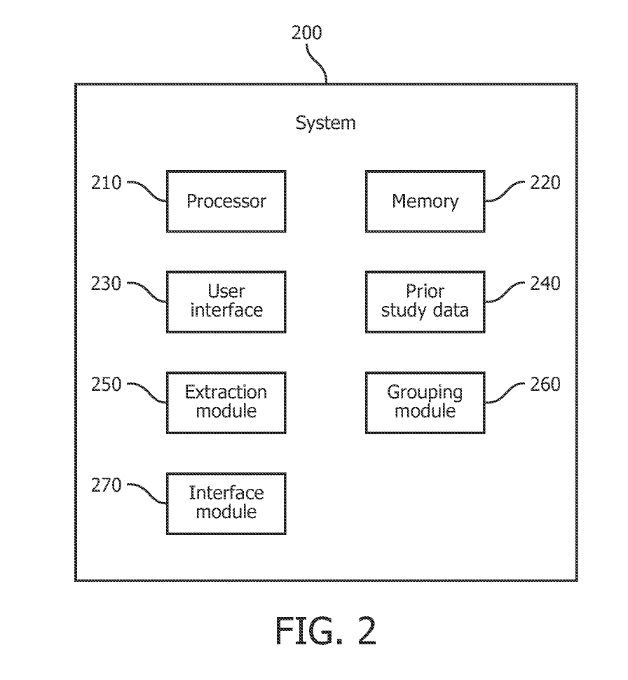

FIG. 2 illustrates an exemplary system 200 for providing a radiologist with information useful to establish context information for a current study. The system 200 may typically be computer-implemented, and may include common elements of a computing system that are known in the art, such as a processor 210, a memory 220, and a user interface 230. The memory 220 may store prior study data 240 for one or more patients, including a current patient whom the radiologist is currently treating. The prior study data 240 may be stored in accordance with the Digital Imaging and Communications in Medicine (“DICOM”) format that will be familiar to those of skill in the art, although this is only exemplary and other formats may alternatively be used. In one common embodiment, the user interface 230 may comprise three displays, with the left display showing a user workspace, the center display showing a current study, and the right display showing a prior study, but it will be apparent to those of skill in the art that this is only exemplary and that other configurations of one or more displays may be possible without departing from the broader principles described herein.

The system 200 also includes exemplary modules, which may be modules of code that are stored in the memory 220 and executed by the processor 210 to perform functions that will be described below with reference to the method 300. These include an extraction module 250 extracting relevant information from the prior study data 240, a grouping module 260 grouping related studies in a predefined or user-specified manner, and an interface module 270 generating a graphical display enabling the radiologist to visualize study groupings in the manner that will be described in further detail below. Those of skill in the art will understand that the delineation of the performance of method 300 as by three separate modules is only exemplary and that the functions may alternately be performed by an integrated software application, or multiple applications having their functions delineated differently from the manner described herein.

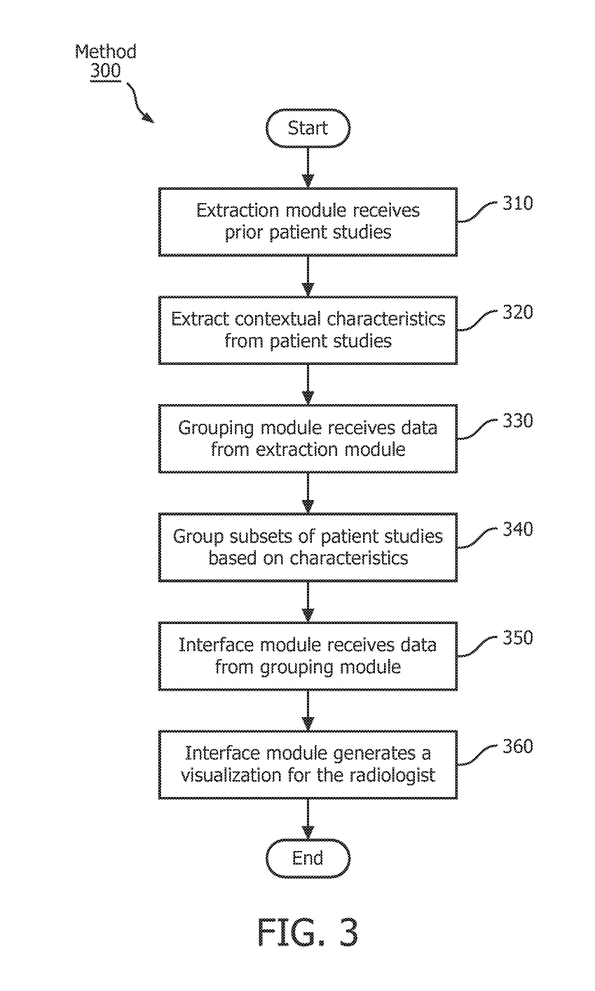

FIG. 3 illustrates a method 300 for generating a rendering to aid a radiologist in the process of establishing context for a current study. Performance of the method 300 may be induced by a radiologist activating the system 200 or instructing the system 200 to display data about a particular patient. In step 310, the extraction module 250 retrieves all of the patient's prior studies from the prior study data 240. This may be accomplished through standard techniques for data retrieval, database querying, etc. As noted above, the data retrieved from the prior study data 240 may be formatted in accordance with the DICOM standard.

In step 320, the extraction module 250 extracts from the patient's prior art studies contextual characteristics of the studies. Characteristics may include body part, reason for exam, modality, etc. The characteristics may be stored in, and the extraction module 250 may extract the characteristics from, both the metadata concerning the studies and the content of the reports, which, as noted above, may comprise text in a narrative format.

As noted above, metadata of the prior studies may commonly be stored in accordance with the DICOM standard. Various characteristics may be extracted from various DICOM attributes (or, as will be apparent to those of skill in the art, other metadata elements when data is stored in a format other than DICOM). For example, a study modality characteristic can be extracted directly from a DICOM attribute and may correspond to DICOM Modality field (0008, 0060). A body part of study characteristic can be extracted directly from a DICOM attribute and may correspond to DICOM Body Part Examined field (0018, 0015).

Some characteristics may be determined by extracting metadata and applying natural language processing (“NLP”), such as using the MetaMap NLP engine, to the extracted text. For example, a reason for exam characteristic can be determined by extracting text from the DICOM tag (0032, 1030) and using NLP techniques to extract diagnostic terms from the narrative text therein. Similarly, an anatomy of study characteristic may be determined by applying NLP techniques to extract a specific body part from narrative descriptions found in the Study Description DICOM tag (0008, 1030), the Protocol Name DICOM tag (0018, 1030), and the Series Description DICOM tag (0008, 103e). It will be apparent to those of skill in the art that the specific characteristics extracted from metadata discussed above are only exemplary, and that other characteristics may be extracted in other embodiments. Continuing with the exemplary embodiment in which metadata is in the DICOM standard, other useful tags may include Procedure Code, Requested Procedure Code, and Scheduled Procedure code.

As noted above, in addition to metadata, the content of the reports, including reason for exam and comparison studies, may be extracted from the narrative text of the prior studies. As described above, an NLP technique may be used to perform this extraction. NLP may be capable of determining sectional structure of the reports, including sections, paragraphs, and sentences. This may include using a maximum entropy classifier that assigns, to each end-of-sentence character (e.g., a period, an exclamation mark, a question mark, a colon, or a backslash-n) one of four labels:

1) The character marks the end of a sentence and the sentence is a section header

2) The character marks the end of a sentence and the sentence ends a paragraph

3) The character marks the end of a sentence and the sentence is neither a section header nor the last sentence of a paragraph

4) The character does not mark the end of a sentence

Section headers may be normalized with respect to five classes: technique, comparison, findings, impressions, and none. As used here, “normalized” means that entries in different reports, the format of which may vary from institution to institution or radiologist to radiologist (e.g., one institution might call the findings section “FINDINGS,” another might call it “FINDING,” while still another might call it “OBSERVATIONS,” etc.), are updated to fit into the standard classes noted above. Other than section headers, sentences may be grouped into paragraphs. The first sentence in each paragraph may be compared against a list of paragraph headers (e.g., “liver”, “spleen”, “lungs”, etc.), and sentences that match an entry in the list are marked as being paragraph headers. In addition to the above, diagnosis-related terms and anatomy-related terms may be extracted from a clinical history section, and dates of comparison studies may be extracted.

In step 330, the grouping module 260 receives the studies and extracted characteristics determined by the extraction module 250 in step 320. This may occur through any standard means for passing data from one computing routine to another. In step 340, the grouping module 260 groups one or more subsets of the studies for subsequent display based on the characteristics corresponding to the studies that comprise the one or more subsets. As will be described hereinafter, the characteristics may be used to group the studies into groups that are related to one another. The grouping may be in a manner that is preconfigured or user-specified. The following describes a variety of exemplary manners for grouping the studies, but it will be apparent to those of skill in the art that other groupings may be possible without departing from the broader principles described herein.

In one exemplary grouping, body part characteristics extracted from the studies may be mapped to organ systems within the human body. By performing such mapping, studies may be grouped by organ and subsequently presented to the radiologist in organ-based groupings. In another exemplary grouping, grouping may be made based on diagnostic terms extracted from “reason for exam” or “clinical history” sections of the reports. This may result in a grouping of prior studies that are related to a same basis for examination.

In another exemplary grouping, characteristics extracted from comparison sections of study reports may be used to group studies that were described as relevant to one another. For example, a comparison section of a report of a given prior study may contain dates of other prior studies that were used for comparison to the given prior study. It will be apparent to those of skill in the art that a prior study may be used and referenced in a report because there is some relationship between the current study and the prior study. Thus, these extracted characteristics may be used to group studies that have an explicit relationship to one another made in the reports.

In another embodiment, prior to grouping, body parts extracted from the reports may be normalized using an ontology such as Systematized Nomenclature of Medicine (“SNOMED”) or Unified Medical Language System (“UMLS”). For example, the knowledge from such an ontology may be used to determine that one study that has an extracted characteristic “kidney” should be grouped with another study having an extracted characteristic “renal”. Similarly, association relationships (e.g., “is-part-of” relationships) contained in such an ontology may be used to determine that two body parts are related and that studies having characteristics of the two body parts should be grouped together. For example, the relationships from such an ontology may be used to determine that a study that has an extracted characteristic “liver” should be grouped with another study having an extracted characteristic “abdomen”.

In another embodiment, a data-driven approach may be used to define a matrix and compare a feature vector of a current study with feature vectors of prior studies. Such a matrix could contain feature vectors from the current study and from prior studies. Each column of the matrix may represent a feature extracted from study metadata such as DICOM tags (e.g., modality, body part 1, body part 2, etc.), as well as words or phrases extracted from the report; each row in the matrix may represent extracted feature information for a single study. Statistical clustering techniques that are known in the art (e.g., using k-means) may then be applied to the various feature vectors to identify groups of studies that are similar.

In step 350, the interface module 270 receives the studies and one or more groupings thereof determined by the grouping module 260 in step 340. As noted above with reference to step 330, this may occur through any standard means for passing data from one computing routine to another. In step 360, the interface module 270 generates a visualization based on the one or more groupings identified by the grouping module 260 and provides the visualization to the radiologist by the user interface 230. In the common three-display embodiment of a user interface 230 described above, the interface module 270 may provide this visualization on the right-hand display.

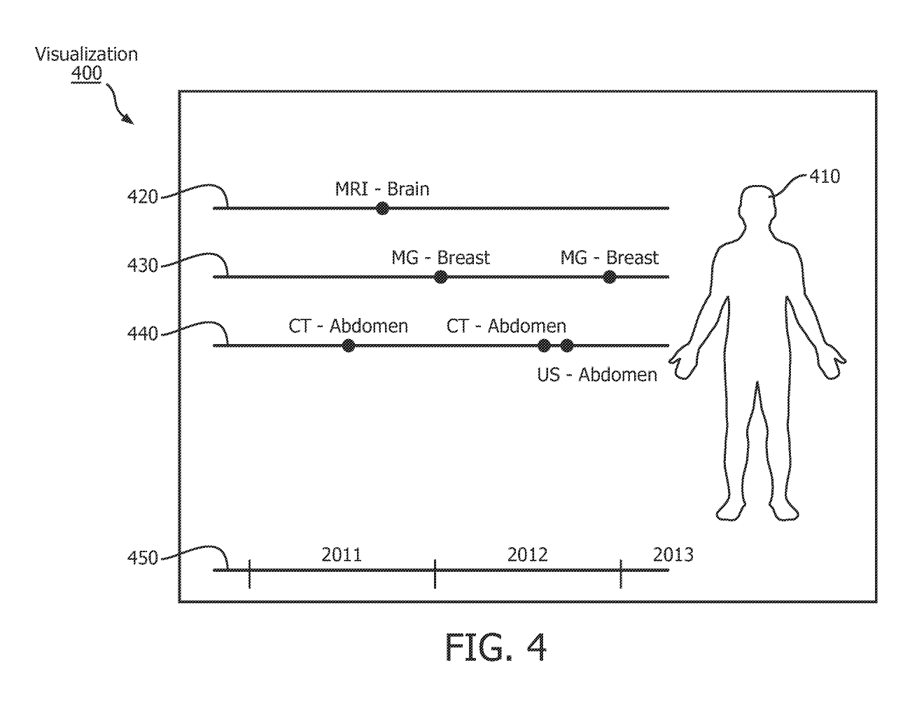

The interface module 270 may display the grouped studies in a variety of specific manners. In one exemplary embodiment, the interface module 270 may provide to the user interface 230 a visualization showing study timelines in conjunction with an illustration of a human. FIG. 4 shows such a visualization 400 including a human 410. The visualization 400 includes a timeline of brain studies 420 next to the head of the human 410, a timeline of breast studies 430 next to the chest of the human 410, and a timeline of abdomen studies 440 next to the abdomen of the human 410. It will be apparent to those of skill in the art that the particular timelines shown in the visualization 400 are only exemplary and that the particular timelines generated may vary depending on the clinical history of the patient for whom the visualization 410 is being prepared. The visualization 400 may also include a time scale 450, to which the timelines 420, 430 and 440 may all be scaled.

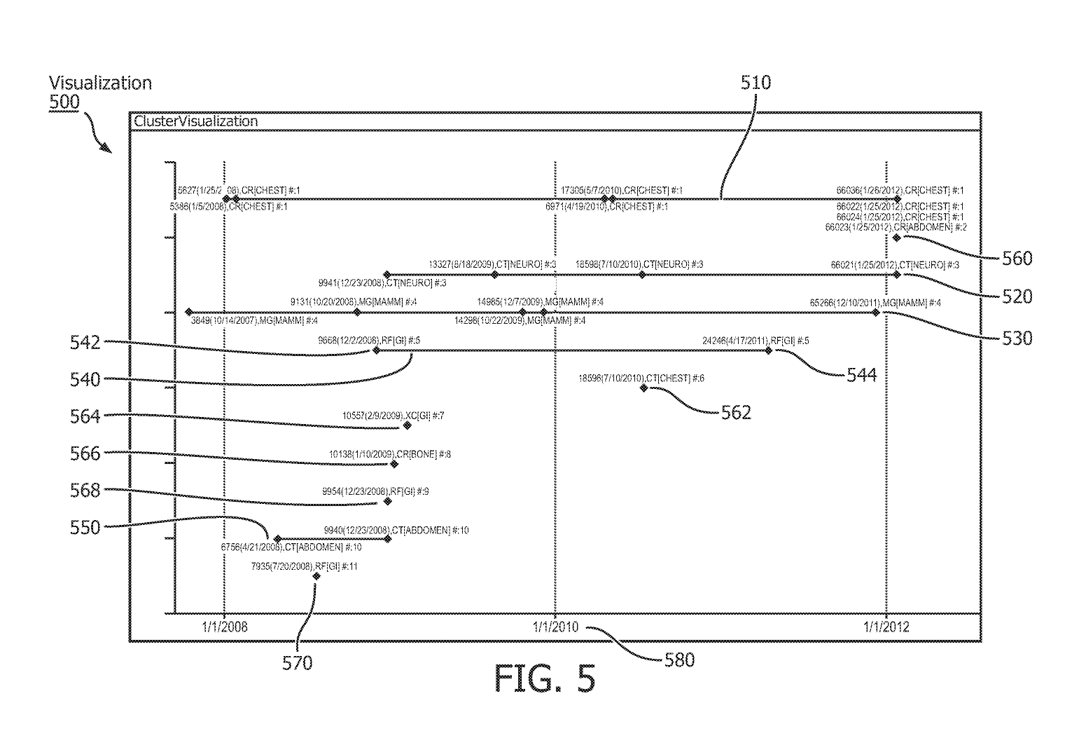

In another exemplary embodiment, the interface module 270 may provide to the user interface 230 a visualization showing study timelines grouped based on explicit references to prior studies. As noted above, this may be accomplished using information extracted from the Comparison sections of study reports. FIG. 5 shows such a visualization 500. The visualization 500 includes timelines 510, 520, 530, 540 and 550, each of which include two or more studies determined in the prior steps to be related to one another based on explicit references to one another. For example, the timeline 540 may include studies 542 and 544, and study 544 may explicitly reference study 542 in its comparison section. The visualization 500 also includes studies 560, 562, 564, 566, 568 and 570 that were not identified as related to one another in the above steps. The timelines 510, 520, 530, 540 and 550 and the ungrouped studies 560, 562, 564, 566, 568 and 570 are displayed along a common time scale 580.

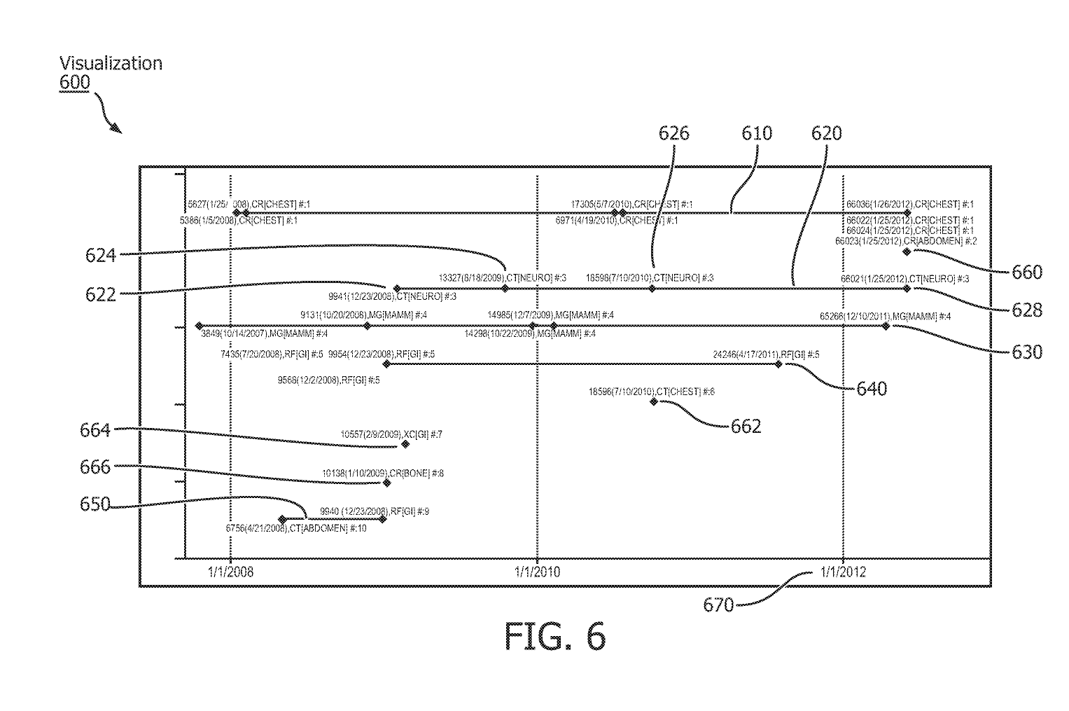

In another exemplary embodiment, the interface module 270 may provide to the user interface 230 a visualization showing study timelines grouped by modality and body part. As noted above, this may be accomplished using information extracted from the Comparison sections of study reports. FIG. 6 shows such a visualization 600, showing the same studies as shown in the visualization 500 of FIG. 5 but grouped in a different manner. The visualization 600 includes timelines 610, 620, 630, 640 and 650, each of which include two or more studies determined in the prior steps to be related to one another based on explicit references to one another. For example, the timeline 620 may include studies 622, 624, 626 and 628, each of which may be a neurological computed tomography (“CT”) scan. The visualization 600 also includes studies 660, 662, 664 and 666 that were not identified as related to one another in the above steps. The timelines 610, 620, 630, 640 and 650 and the ungrouped studies 660, 662, 664 and 666 are displayed along a common time scale 670.

As noted above, the visualization 600 shows the same studies as the visualization 500 of FIG. 5 grouped differently. For example, ungrouped study 568 of FIG. 5, a gastrointestinal (“GI”) radio frequency (“RF”) scan, is grouped into timeline 640 of FIG. 6. It will be apparent to those of skill in the art that this grouping in the visualization 600 may be due to the fact that the timeline 640 includes a grouping of GI RF scans. However, the study 568 may be omitted from a timeline in the visualization 500 due to the lack of an explicit reference thereto in other studies (e.g., those comprising timeline 540 of visualization 500), the criteria used for grouping studies in visualization 500.

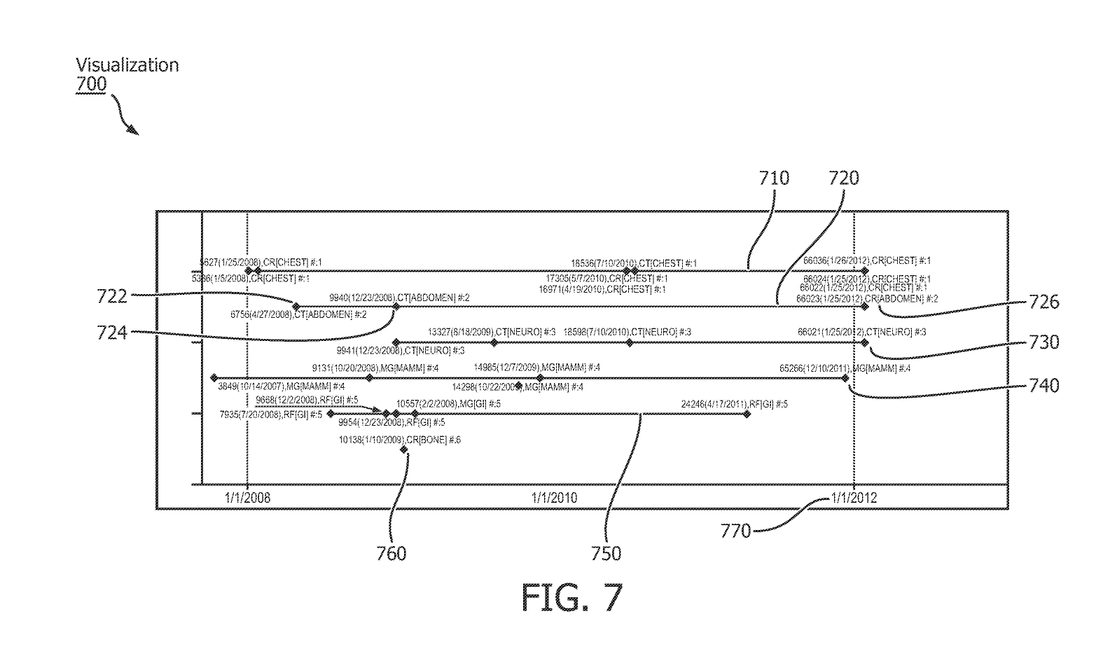

In another exemplary embodiment, the interface module 270 may provide to the user interface 230 a visualization showing study timelines grouped by body part without regard to modality. As noted above, this may be accomplished using information extracted from the Comparison sections of study reports. FIG. 7 shows such a visualization 700, showing the same studies as shown in the visualization 500 of FIG. 5 and the visualization 600 of FIG. 6 but grouped in a different manner. The visualization 700 includes timelines 710, 720, 730, 740 and 750, each of which include two or more studies determined in the prior steps to be related to one another based on explicit references to one another. For example, the timeline 720 may include studies 722, 724 and 726, each of which may be an abdominal scan, with studies 722 and 724 being abdominal CT scans and study 726 being an abdominal computed radiography (“CR”) scan. The visualization 700 also includes study 760 that was not identified as related to any other studies in the above steps. The timelines 710, 720, 730, 740 and 750 and the ungrouped study 760 are displayed along a common time scale 770.

As noted above, the visualization 700 shows the same studies as the visualization 500 of FIG. 5 and the visualization 600 of FIG. 6 grouped differently. For example, ungrouped study 662 of FIG. 6, a chest CT scan, is grouped into timeline 710 of FIG. 7. It will be apparent to those of skill in the art that this grouping in the visualization 700 may be due to the fact that the timeline 710 includes a grouping of chest scans without regard to modality. However, the study 662 may be omitted from a timeline in the visualization 600 due to its different modality from the studies comprising timeline 610, the criteria used for grouping studies in visualization 600.

It will be apparent to those of skill in the art that the visualizations 400, 500, 600 and 700 described above are only exemplary, and that other criteria for study grouping may be used without deviating from the broader principles of the exemplary embodiments. The user interface 230 may also enable the radiologist to correct or update study associations using a “drag and drop” or other interface. For example, a radiologist viewing the visualization 600, including timeline 610 and ungrouped study 662, may elect to associate study 662 with timeline 610; it will be apparent to those of skill in the art that this will result in a timeline similar to timeline 710 of visualization 700. Additionally, the radiologist may interact with the user interface 230 to select one or more of the studies (e.g., a single study, a portion of a selected timeline, an entire selected timeline, a plurality of selected timelines, etc.) and launch the studies for interpretation.

The visualizations that may be provided by the exemplary embodiments may aid a radiologist in establishing clinical context for a current study in two ways. First, the study groupings themselves may enable the radiologist to gain an overall understanding of the patient's history by providing a general overview of the type of scans that have been conducted on the patient over a desired time interval. Second, because the studies may be presented to the radiologist in grouped subsets rather than wholesale as shown in FIG. 1, it may be easier for the radiologist to identify and select a desired one or more of the reports for retrieval and further review prior to performing a current study.

Those of skill in the art will understand that the above-described exemplary embodiments may be implemented in any number of manners, including as a software module, as a combination of hardware and software, etc. For example, the exemplary method 300 may be embodied in a program stored in a non-transitory storage medium and containing lines of code that, when compiled, may be executed by a processor. Additionally, it will be apparent to those of skill in the art that though this disclosure makes reference to specific types of medical imaging studies, the broader principles described herein may be equally applicable to any type of medical imaging study known to those of skill in the art. This may include x-ray studies or other types of radiographic studies, RF studies, CT studies, CR studies, magnetic resonance imaging (“MRI”) studies, ultrasound studies, position emission tomography (“PET”) studies or other types of nuclear imaging studies, photoacoustic studies, thermographic studies, echocardiographic studies, functional near-infrared spectroscope (“FNIR”) studies, or any other type of medical imaging study not expressly mentioned herein.

It will be apparent to those skilled in the art that various modifications may be made to the exemplary embodiments, without departing from the spirit or the scope of the invention. Thus, it is intended that the present invention cover modifications and variations of this invention provided they come within the scope of the appended claims and their equivalents.

1. A method, comprising:
receiving a plurality of reports, each of the reports describing a corresponding one of a plurality of medical imaging studies of a patient;
extracting, from each of the reports, a corresponding characteristic;
identifying a subset of the reports based on a similarity of the characteristics of the reports comprising the subset; and
generating a visualization of a portion of a patient history for the patient, the portion comprising the subset of the reports
wherein the extracting of the corresponding characteristic from each of the reports includes extracting the characteristic from narrative text of the report by means of natural language processing, and
wherein the identifying of a subject of the reports comprises grouping the subset of the reports
using a medical ontology.

2. The method of claim 1, wherein the visualization comprises a plurality of timelines of medical imaging studies for the patient.

3. The method of claim 2, wherein each of the plurality of timelines comprises one of medical imaging studies having a same body part, medical imaging studies having a same body part and modality, and medical imaging studies having explicit references to one another.

4. The method of claim 2, wherein the plurality of timelines are shown in relation to an illustration of a human body and/or are shown with relation to a common time scale.

5. (canceled)

6. The method of claim 2, wherein the visualization further comprises an indication of one of the medical imaging studies that is not part of one of the timelines.

7. The method of claim 1, wherein a plurality of corresponding characteristics are extracted from each of the reports.

8. The method of claim 1, wherein the characteristic comprises one of a modality, a body part, a study description, a protocol name, a series description, a reason for study and a procedure code.

9. The method of claim 1, wherein the extracting of the corresponding characteristic of each of the studies includes extracting from metadata of each of the studies.

10. The method of claim 9, wherein the metadata is formatted in accordance with the Digital Imaging and Communications in Medicine standard.

11. (canceled)

12. (canceled)

13. The method of claim 1, wherein the medical ontology comprises one of Systematized Nomenclature of Medicine and Unified Medical Language System.

14. A system, comprising:
a non-transitory memory storing a plurality of reports, each of the reports describing a corresponding one of a plurality of medical imaging studies of a patient;
a processor executing:
an extraction module extracting, from each of the reports, a corresponding characteristic;
a grouping module identifying a subset of the reports based on a similarity of the characteristics of the reports comprising the subset; and
a visualization module generating a visualization of a portion of a patient history for the patient, the portion comprising the subset of the reports; and
a graphical user interface displaying the visualization to a user of the system
wherein the extracting of the corresponding characteristic from each of the reports includes extracting the characteristic from narrative text of the report by means of natural language processing, and
wherein the identifying of a subset of the reports comprises grouping the subset of the reports using a medical ontology.

15. The system of claim 14, wherein the medical imaging studies comprise one of radiographic studies, radio frequency studies, computed tomography studies, computed radiography studies, magnetic resonance imaging studies, ultrasound studies, position emission tomography studies, nuclear imaging studies, photoacoustic studies, thermographic studies, echocardiographic studies, and functional near-infrared spectroscope studies.

16. The system of claim 14, wherein the visualization comprises a plurality of timelines of medical imaging studies for the patient.

17. (canceled)

18. (canceled)

19. The system of claim 14, wherein the extraction module is further arrange for extracting the corresponding characteristic of each of the studies from metadata of each of the studies.

20. A non-transitory computer-readable storage medium storing a set of instructions executable by a processor, the set of instructions, when executed by the processor, causing the processor to perform operations comprising:
receiving a plurality of reports, each of the reports describing a corresponding one of a plurality of medical imaging studies of a patient;
extracting, from each of the reports, a corresponding characteristic;
identifying a subset of the reports based on a similarity of the characteristics of the reports comprising the subset; and
generating a visualization of a portion of a patient history for the patient, the portion comprising the subset of the reports,
wherein the extracting of the corresponding characteristic from each of the reports includes extracting the characteristic from narrative text of the report by means of natural language processing, and
wherein the identifying of a subset of the reports comprises grouping the subset of the reports using a medical ontology.
